(19) United States
(12) Patent Application Publication
(10) Pub. No.: US 2004/0111061 A1
(43) Pub. Date: Jun. 10, 2004

(54) Trocar having an inflatable cuff for maintaining an insufflated abdominal cavity during an open laparaoscopy procedure

(76) Inventor: Diana Curran, Omaha, NE (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/706,581

(22) Filed: Nov. 12, 2003

Related U.S. Application Data
(60) Provisional application No. 60/425,476, filed on Nov. 12, 2002.

(51) Int. Cl. A61M 5/32

(52) U.S. Cl. 604/174

(57) ABSTRACT

An inflatable cuff for use with a trocar inserted within an abdominal cavity. The trocar exhibits a hollowed interior and includes an upper funnel portion and an interconnecting and downwardly extending sleeve portion. The cuff exhibits a flexible and inflatable annular shape and is constructed of a fluid-tight material. The cuff secures in an initially deflated condition at a location about the sleeve portion of the trocar. A fluid line extends through a port in the trocar and to a location along the cuff, the cuff being inflated after insertion of the sleeve within a patient's abdominal cavity to maintain a sealed condition within the abdominal cavity.

### CROSS REFERENCE TO RELATED APPLICATIONS

The present application claims the priority of Provisional Application Serial No. 60/425,476, filed Nov. 12, 2002, and entitled “Trocar Having an Inflatable Cuff for Maintaining an Insufflated Abdominal Cavity During an Open Laparoscopy Procedure.”

### BACKGROUND OF THE INVENTION

1. Field of the Invention

The present invention relates generally to open laparoscopic procedures employing a trocar. More particularly, the present invention is directed to a modification and improvement of a trocar which employs an inflatable cuff surrounding a sleeve portion of the trocar and which, upon insertion of the trocar into a patient's abdominal cavity which has been insufflated with CO₂, acts to maintain the sealing condition within the cavity during the performance of a medical procedure.

2. Description of the Prior Art

A number of medical procedures are known in the prior art which employ the use of a trocar for assisting in open abdominal surgical procedures. A trocar is most generally defined as a sharp pointed rod which fits inside a tube and is used to pierce the skin and wall of a cavity or canal within the body and in order to inject or vacuum out raw fluids, to insert drugs or solutions, or to guide the placement of a soft tube or catheter. In certain applications, the trocar is removed after insertion of the tube, which is left in place.

A particular medical procedure is known as a laparoscopic procedure and involves inserting the trocar through a dissection made through the skin, subcutaneous tissue, muscle and peritoneum and into the abdominal cavity of the patient. Stabilizing sutures are then placed through the fascia to attach the sleeve of the trocar in place against the abdominal cavity wall.

One known laparoscopic technique is known as a “blind technique”, while another “open technique” involves insufflating the patient's abdominal cavity with such as a CO₂ charge to create a pneumoperitoneum condition which allows the surgeon to operate within the abdomen with the patient's bowels out of the way. One problem associated with the open technique is in maintaining the CO₂ seal because of inherent leakage occurring around the trocar sleeve.

Relevant examples drawn from the prior art include U.S. Pat. No. 5,628,732, issued to Antoon, Jr. et al., which teaches a trocar having a universal seal for sealing against surgical instruments of varying diameter and in order to maintain an insufflated condition in a body cavity. The universal seal has an elastomeric sealing component with a centrally located interior region containing an aperture, and a concentrically located sealing region. The sealing region is composed of an integral laminate having an overlaying layer co-molded with an underlying layer. The co-molded laminate balances the properties of tear resistance and elasticity and which are necessary for a working universal seal, and does so without the need for resilient legs or protectors to facilitate the opening of the aperture of the seal or to prevent tearing when instruments are used.

U.S. Pat. No. 5,147,316, to Castillenti, discloses a laparoscopic trocar exhibiting a sleeve which may be selectively fixed to an abdominal wall by a bumper in cooperation with a balloon mounted on the distal end of the sleeve. Following insertion of the trocar and the distal end of the sleeve, the trocar is withdrawn and the balloon inflated by a syringe through the distensible coupling and a balloon inflation duct. The sleeve includes ratchets on its outer surface, and the bumper exhibits a ratchet tool in its inner channel and which ensures that the bumper will not move backwards along the sleeve and which may further be released and the balloon deflated to permit removal of the sleeve from the abdomen.

Finally, U.S. Pat. No. 5,941,852, issued to Dunlap et al., teaches a cannula converter for use with a trocar assembly used in surgical procedures, such as appendectomies, and for maintaining a sealed working channel in a body wall and an obturator which creates the working channel through the body wall while protecting patients and medical personnel from harm. A cannula converter can be employed from the trocar to enable a surgeon to use surgical instruments having a smaller outer diameter than the inner diameter of the cannula without deflating a body cavity. A site stabilizer is also discussed and which can be used with the trocar to prevent the cannula from being inadvertently withdrawn from the body cavity during a surgical procedure.

### SUMMARY OF THE PRESENT INVENTION

The present invention discloses an inflatable cuff for use with a trocar sleeve. The trocar exhibits an upper funnel portion and a lower and interconnected sleeve portion. A substantially doughnut-shaped cuff surrounds the base of the sleeve.

Upon insertion of the sleeve portion of the trocar within the abdominal cavity, sutures are applied between the upper funnel portion of the trocar and the area of the abdominal wall surrounding the trocar. The cuff is maintained in an initially deflated condition upon insertion and is subsequently inflated in order to create a sealed condition about the trocar at the underside of the patient's peritoneum.

A known and popular surgical technique is further the insufflating of a patient's abdominal cavity, such as utilizing a carbon dioxide gas, and in order to provide the surgeon with an unobstructed view of the patient's cavity. The advantage of the inflatable cuff is that it maintains a sealed condition of the insufflated abdominal cavity, thus decreasing the surgeon's frustrations as well as operating time by avoiding the required waiting period for the pneumoperitoneum (insufflated) condition to be reestablished.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 illustrates a first dissected, inserted and pre-inflated condition of the trocar cuff, inserted within the patient's abdominal cavity and according to a preferred embodiment of the present invention;

FIG. 2 illustrates a succeeding and inflated condition of the trocar cuff according to the present invention;

FIG. 3 is an environmental view illustrating one laparoscopic procedure employing the trocar and inflatable cuff and which in particular shows a camera inserted through the open interior of the trocar tube;

FIG. 4 is a view of the inflatable cuff and which is capable of being retrofitted with any size of disposable trocar according to the present invention; and

FIG. 5 is an illustration of an inflatable cuff in use with a trocar and according to a further preferred variant of the present invention.

### DETAILED DESCRIPTION OF THE PREFERRED EMBODIMENTS

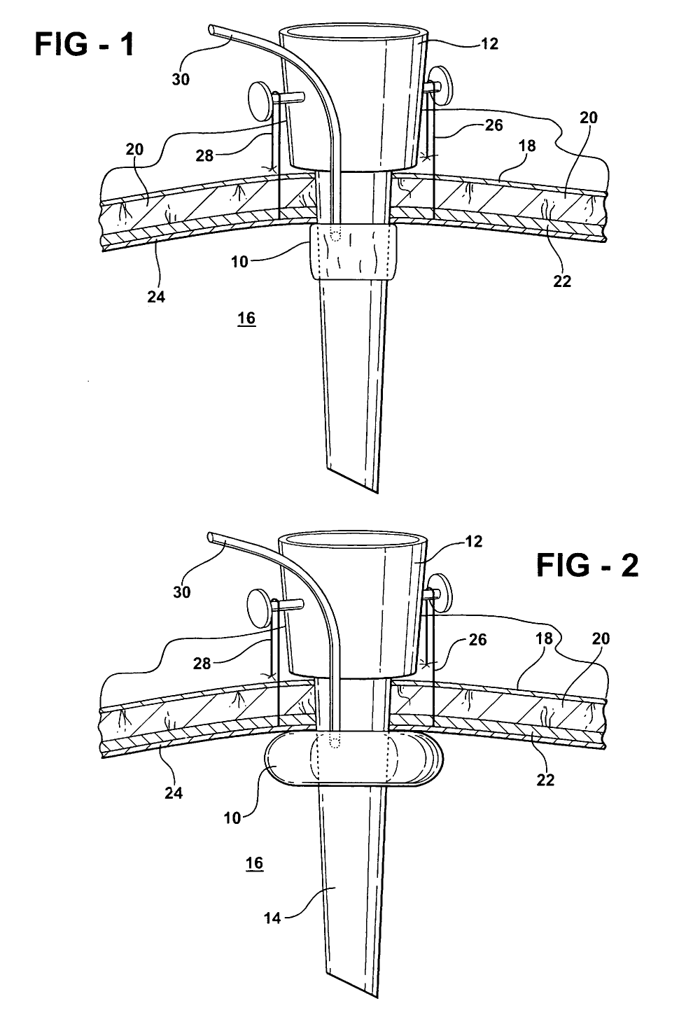

Referring to FIG. 1, a first dissected, inserted and pre-inflated condition of a cuff 10 in use with a trocar is illustrated according to a preferred embodiment of the present invention. As explained previously, the trocar (and referencing in the variant of FIG. 1 features an upper funnel portion 12 and lower and interconnected sleeve portion 14) is constructed of a durable polymer or other suitable and hygienic material and is inserted within the patient's abdominal cavity 16, such again occurring through the dissection of the skin 18, subcutaneous tissue 20, muscle 22 and peritoneum 24 layers with a scalpel or other suitable cutting instrument (not shown).

The trocar sleeve 14 is then inserted through the dissected layers and sutures 26 and 28 are applied between the upper funnel portion 12 of the trocar, see additionally lateral button projections 29, and the area of the patient's abdominal wall (see again layers 18, 20, 22, and 24) surrounding the trocar. A port or passageway 30 is secured to the trocar, such as extending from a first inlet location proximate the upper funnel portion 12 to an outlet location proximate a location of the lower sleeve portion 14, which is in communication with the inflatable cuff 10. As again is illustrated in FIG. 1, the cuff 10 (usually constructed of a flexible and airtight material such as a thin rubber or the like) is initially provided in a deflated condition in order to be positioned at the underside location of the patient's peritoneum 24 (this again defining the inner wall surface of the patient's abdomen).

Upon subsequent reference to FIG. 2, a further inflated condition of the encircling trocar cuff 10 is illustrated. The cuff 10 typically is constructed of a plasticized/polymerized, flexible and air impermeable material and into which is introduced a pressurized fluid (such as typically being a gaseous O₂) fed through the passageway 30 and in order to expand and seal the cuff 10 against the underside of the peritoneum. In this fashion, and as has been previously described, the cuff serves to seal the inner edges of the abdominal wall dissection surrounding the inserted trocar sleeve and to thereby maintain the insufflated condition within the abdominal cavity.

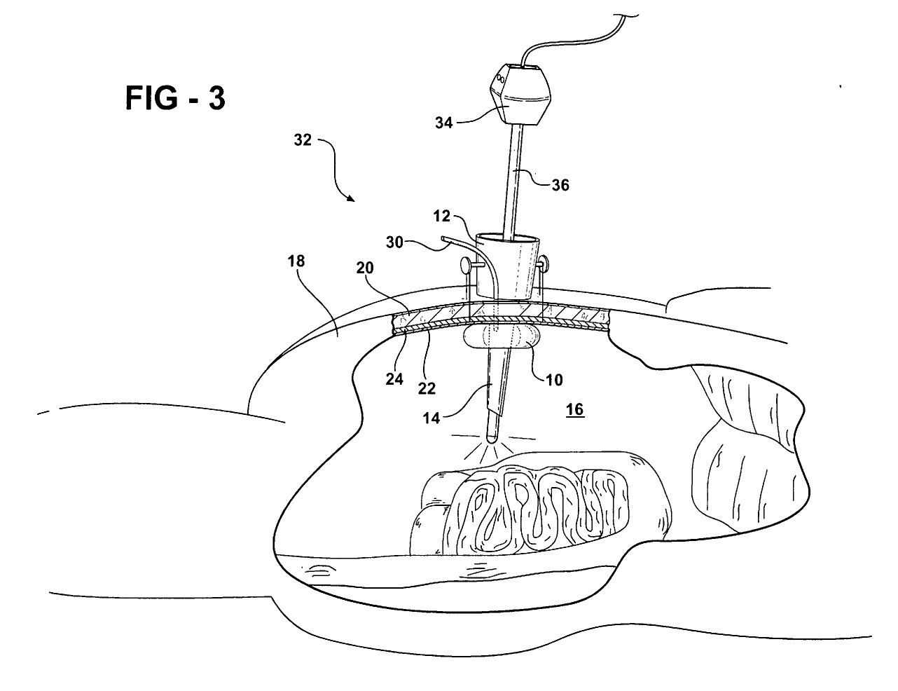

Referring to FIG. 3, an environmental view is generally illustrated at 32 of a laparoscopic procedure employing the trocar and inflatable cuff. In particular, FIG. 3 shows a camera 34 applied in combination with a laparoscope 36 and inserted through the open interior of the trocar sleeve. As again has been previously described, the advantage of having the inflatable cuff 10 maintain the insufflated condition of the abdominal cavity 10 is to afford the surgeon an unobstructed view of the patient's cavity and without interference of the bowels and the like.

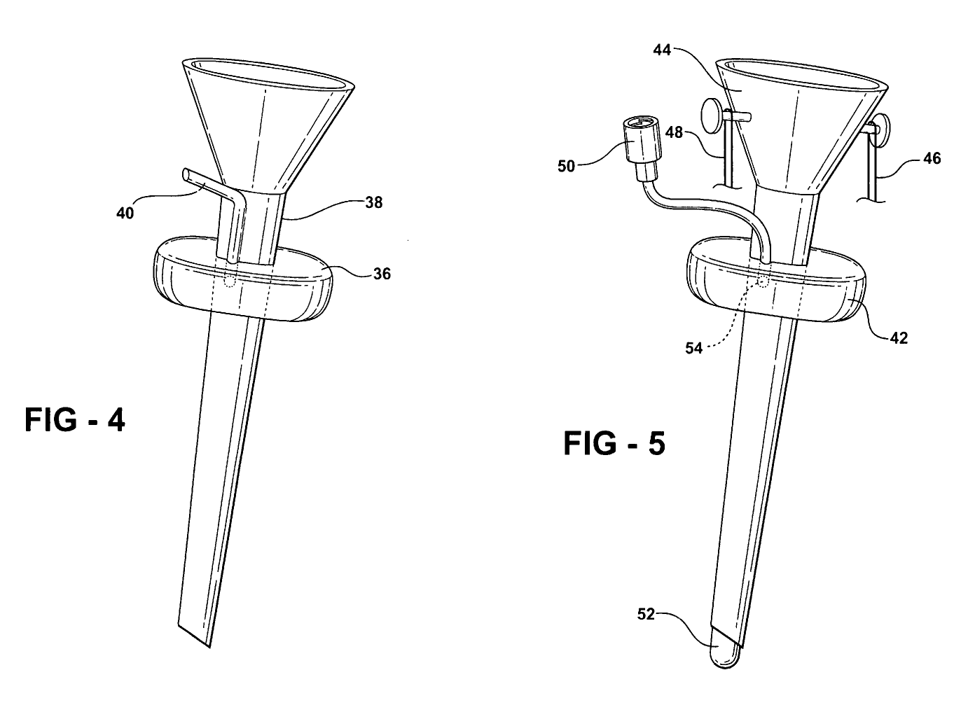

FIG. 4 illustrates at 36 a view of an inflatable cuff 36 and which is capable of being retrofitted with any size of disposable trocar, see also illustrated at 38, according to the present invention. As with the previous variants disclosed, the trocar 38 includes a port 40 for inflating (or insufflating) the cuff 36. It is also understood that the cuff 36 is capable of being utilized with any size of trocar, such including those exhibiting diameters of 5 mm, 7 mm, 8 mm, 10 mm, 12 mm, and upward.

Finally, and referring to FIG. 5, an illustration is shown of an inflatable cuff 42 according to a yet further modification and in use with a likewise further modified trocar 44. The trocar 44 in this variant includes anchoring sutures 46 and 48, as well as a modified port 50 for insufflating the cuff after insertion within the abdomen.

The variant of FIG. 5 also contemplates the terminating sleeve end of the trocar exhibiting a blunt tip 52 and the inflated cuff 42 further exhibiting a peephole 54 for an associated camera (not shown). Also, the port 50 for insufflating the cuff 42 can also be provided with an IV (intravenous) type tubing, such as having a 4-5 cm length and a Luer lock end, this providing the cuff with operating characteristics similar to that of an endotracheal tube.

Having described my invention, additional preferred embodiments will become apparent to those skilled in the art to which it pertains and without deviating from the scope of the appended claims:

I claim:

1. An inflatable cuff for use with a trocar inserted within an abdominal cavity, the trocar exhibiting a hollowed interior and including an upper funnel portion and an interconnecting and downwardly extending sleeve portion, said cuff comprising:
said cuff exhibiting a flexible and inflatable annular shape and being constructed of a fluid-tight material;
said cuff securing in an initially deflated condition at a location about the sleeve portion of the trocar, said cuff being subsequently inflated after insertion of said sleeve within a patient's abdominal cavity and insufflation of the cavity, said cuff maintaining a sealed condition within the abdominal cavity during insertion of the trocar.

2. The inflatable cuff according to claim 1, further comprising a port extending through said trocar from a first location outside of the patient's abdominal cavity to a second location in communication with said cuff, a fluid line extending through said port to said cuff and communicating a pressurized gas to inflate said cuff.

3. The inflatable cuff according to claim 1, further comprising said cuff capable of being retrofitted to a variety of different trocar devices.

4. The inflatable cuff according to claim 1, the trocar having a specified shape and size, further comprising a laparoscope inserting through an open interior of the trocar and into the abdominal cavity.

5. The inflatable cuff according to claim 2, further comprising a modified port located at a first extending end of said fluid line, said port capable of receiving an intravenous type tubing in order to provide said cuff and trocar with operating characteristics similar to that of an endotracheal tube.

6. An inflatable cuff for use with a trocar inserted within an abdominal cavity, the trocar exhibiting a hollowed interior and including an upper funnel portion and an interconnecting and downwardly extending sleeve portion, said cuff comprising:
said cuff exhibiting a flexible and inflatable annular shape and being constructed of a fluid-tight material;
said cuff securing in an initially deflated condition at a location about the sleeve portion of the trocar, a fluid line extending through a port in said trocar and to a location along said cuff, said cuff being inflated after insertion of said sleeve within a patient's abdominal cavity to maintain a sealed condition within the abdominal cavity.
